(19) United States
(12) Patent Application Publication
(10) Pub. No.: US 2019/0076037 A1
(43) Pub. Date: Mar. 14, 2019

(54) MICRO AND MACRO ACTIVITY DETECTION AND MONITORING

(71) Applicant: QUALCOMM Incorporated, San Diego, CA (US)

(72) Inventors: Sandip BHARATI, San Diego, CA (US); Soumya DAS, San Diego, CA (US); Ashutosh AGGARWAL, San Diego, CA (US)

(21) Appl. No.: 15/701,191

(22) Filed: Sep. 11, 2017

(51) Int. Cl. A61B 5/024; A61B 5/0205; A61B 5/11; A61B 5/00; G01K 13/00

(52) U.S. Cl. CPC A61B 5/02438; A61B 5/02055; A61B 5/1112; G06Q 10/063114; A61B 5/681; G01K 13/002; A61B 5/1118

(57) ABSTRACT

Systems, methods, apparatuses, and non-transitory computer-readable media for micro and macro activity detection and monitoring are disclosed. One example method includes receiving a profile from a health care provider, the profile comprising a physiological threshold; iteratively during a first time interval: receiving first sensor signals from a first sensor, the first sensor disposed within a first device worn by an individual, receiving second sensor signals from a second sensor, the second sensor disposed within a second device, and determining, using a trained machine learning technique for the individual, and accumulating a macro activity and a micro activity based on the first and second sensor signals, using the accumulated macro and micro activities, determining an aggregate macro activity and an aggregate micro activity for the first time interval; and responsive to determining, using the aggregate macro activity and the aggregate micro activity, that the physiological threshold has been reached, outputting a notification indicating the physiological threshold.

### BACKGROUND

Recently, individuals have been increasingly using personal devices to monitor activity information, such as steps taken during the course of a day. Such information is typically determined using accelerometers to identify steps taken, which are then summed over the course of the day. Such devices may measure other types of information as well, such as number of flights of stairs climbed, pulse rate, and estimated calories burned.

### BRIEF SUMMARY

Various examples are described for micro and macro activity detection and monitoring. One example method includes receiving a profile from a health care provider, the profile comprising a physiological threshold; iteratively during a first time interval: receiving first sensor signals from a first sensor, the first sensor disposed within a first device worn by an individual, receiving second sensor signals from a second sensor, the second sensor disposed within a second device, and determining, using a trained machine learning technique for the individual, and accumulating a macro activity and a micro activity based on the first and second sensor signals, using the accumulated macro and micro activities, determining an aggregate macro activity and an aggregate micro activity for the first time interval; and responsive to determining, using the aggregate macro activity and the aggregate micro activity, that the physiological threshold has been reached, outputting a notification indicating the physiological threshold.

One example device includes a first sensor; a non-transitory computer-readable medium; and a processor in communication with the first sensor and a second sensor, the processor configured to: receive one or more profiles from a health care provider, the profile comprising a physiological threshold; iteratively during a first time interval: receive first sensor signals from the first sensor, receive second sensor signals from a second sensor, and determine, using a trained machine learning technique for an individual associated with the device, and accumulate a macro activity and a micro activity based on the first and second sensor signals, using the accumulated macro and micro activities, determine an aggregate macro activity and an aggregate micro activity for the first time interval; and responsive to a determination, using the aggregate macro activity and the aggregate micro activity, that the physiological threshold has been reached, output a notification indicating the physiological threshold.

One example non-transitory computer-readable medium includes processor-executable program code configured to cause a processor to receive one or more profiles from a health care provider, the profile comprising a physiological threshold; iteratively during a first time interval: receive first sensor signals from a first sensor, the first sensor disposed within a first device worn by an individual, receive second sensor signals from a second sensor, the second sensor disposed within a second device, and determine, using a trained machine learning technique for the individual, and accumulate a macro activity and a micro activity based on the first and second sensor signals, using the accumulated macro and micro activities, determine an aggregate macro activity and an aggregate micro activity for the first time interval; and responsive to a determination, using the aggregate macro activity and the aggregate micro activity, that the physiological threshold has been reached, output a notification indicating the physiological threshold.

One example apparatus includes means for receiving one or more profiles from a health care provider, the profile comprising a physiological threshold; means for receiving first sensor signals from a first sensor, the first sensor disposed within a first device worn by an individual, means for receiving second sensor signals from a second sensor, the second sensor disposed within a second device, and means for determining, using a trained machine learning technique for the individual, and accumulating a macro activity and a micro activity based on the first and second sensor signals during a first time interval, means for determining an aggregate macro activity and an aggregate micro activity for the first time interval using the accumulated macro and micro activities; and means for determining, using the aggregate macro activity and the aggregate micro activity, that the physiological threshold has been reached; and means for outputting a notification indicating the physiological threshold.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIGS. 1 and 2 show example systems for micro and macro activity detection and monitoring;

FIG. 3 shows an example computing device for micro and macro activity detection and monitoring;

FIG. 4 shows an example wearable computing device for micro and macro activity detection and monitoring; and

FIG. 5 shows an example method for micro and macro activity detection and monitoring.

### DETAILED DESCRIPTION

Examples are described herein in the context of micro and macro activity detection and monitoring. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

In one illustrative example, a patient is undergoing physical therapy following abdominal surgery and has been assigned an exercise regimen by a physical therapist, but has also been given other restrictions by her physician on the types of exercise she may engage in and limits on the intensities of those exercises. In this example, the patient has been instructed that she may jog, but may not exceed a six mile-per-hour pace (i.e., ten-minute miles) and may not jog for more than ten minutes at a time before resting or walking for at least fifteen minutes. In addition, her physician has instructed her to not perform any high-intensity exercise of any kind for any longer than five minutes at a time, and that after such exertion she needs to rest for at least thirty minutes. The physician then generates a profile for the patient and transmits it to a physiological monitoring app installed on the patient's smartphone

However, her physical therapist has created an exercise regimen for her to help regain strength and conditioning following her surgery. In this example, the exercise regimen includes a daily routine of a thirty minute walk on the treadmill or thirty minutes on an exercise bicycle at a low intensity. In addition, she has been instructed to jog at least twice per week at a moderate intensity level for at least two miles. The physical therapist also provides a profile for the patient and transmits it to the app.

After visiting her physician and physical therapist, she returns home and the next day begins exercising according to the physical therapists instructions. She arrives at her local gym with her smartphone and wearing her smartwatch. She logs into the physiological monitoring app and begins walking on the treadmill. The smartphone detects several nearby Bluetooth (“BT”) beacons and connects to the one with the highest signal strength. In the meantime, her smartwatch continuously monitors her pulse and arm motion. Her smartphone obtains her location using a Global Navigation Satellite System (“GNSS”) receiver, such as a Global Positioning System (“GPS”) receiver. As she walks on the treadmill, the physiological monitoring app obtains location information from the GNSS receiver, information about the BT beacon, accelerometer information from the smartphone's accelerometers, and pulse information and accelerometer information from the smartwatch.

In this example, the physiological monitoring app includes a machine learning (“ML”) model that has been trained using sensor information obtained from the patient's smartphone and smartwatch. Thus, the ML model has been individualized for the patient. The ML model, when operating outside of a training mode, can accept different types of sensor inputs and outputs two different types of activity information: a macro activity and a micro activity. In this example, the macro activity relates to the intensity of detected activity, such as low intensity, moderate intensity, and high intensity. In contrast, the micro activity relates to a specific activity being performed, such as walking, running, sitting, biking, etc. Further, the ML model outputs these micro and macro activities every time it is executed, which may be at a periodic or aperiodic rate. Thus, over the course of an hour, for example, the ML model may output thousands of determined micro and macro activities, such as if it is executed once per second.

The app repeatedly provides samples of sensor data to the trained ML module, including the location information from the GNSS receiver, as well as the pulse information and the accelerometer information from both the smartphone and the smartwatch. For each sample of information provided to the trained ML model, the model determines a macro activity and a micro activity. For example, as the patient walks on the treadmill, the app samples and provides location information, pulse information, and accelerometer information from both the smartwatch and the smartphone, to the trained ML model every second. For each set of sampled information, the model outputs the patient's activity, such as low-intensity walking or moderate intensity jogging.

As the outputs from the model accumulate, the app determines the patient's micro and macro activities and their respective durations. The app then compares these against the profiles provided by the patient's physician and physical therapist. If the app detects that the user's intensity level has been too high for too long, or has been jogging for longer than allowed by the physician, it causes the smartphone to output a warning notification to the patient to reduce the intensity level or to stop jogging. In this example, the app also causes the smartphone to display a timer to indicate how long the patient needs to wait before jogging again or before increasing the intensity level again. In addition, the app logs the deviation from the physician's profile.

As the patient continues to work out on the treadmill, the app monitors progress against the physical therapist's profile to confirm that the patient is performing the prescribed exercise regimen based on the determined macro and micro activities. For example, as the patient walks on the treadmill, the app determines, based on the accumulating micro and macro activities, that the patient is walking at low intensity, and credits the time spent walking against the profile provided by the physical therapist. In addition, the app determines whether the intensity level, based on the determined macro activity, satisfies the profile provided by the physician. If a deviation is detected, or if the patient is approaching a predetermined limit or threshold, the app outputs a notification to the patient.

Thus, the user's smartphone is able to continuously monitor the patient's activity and ensure compliance with programs provided by the patient's health care provider, such as the physical therapist in this example, as well as to ensure the user complies with restrictions or requirements imposed by the patient's physician (also a health care provider).

This illustrative example is given to introduce the reader to the general subject matter discussed herein and the disclosure is not limited to this example. The following sections describe various additional non-limiting examples and examples of systems and methods for micro and macro activity detection and monitoring.

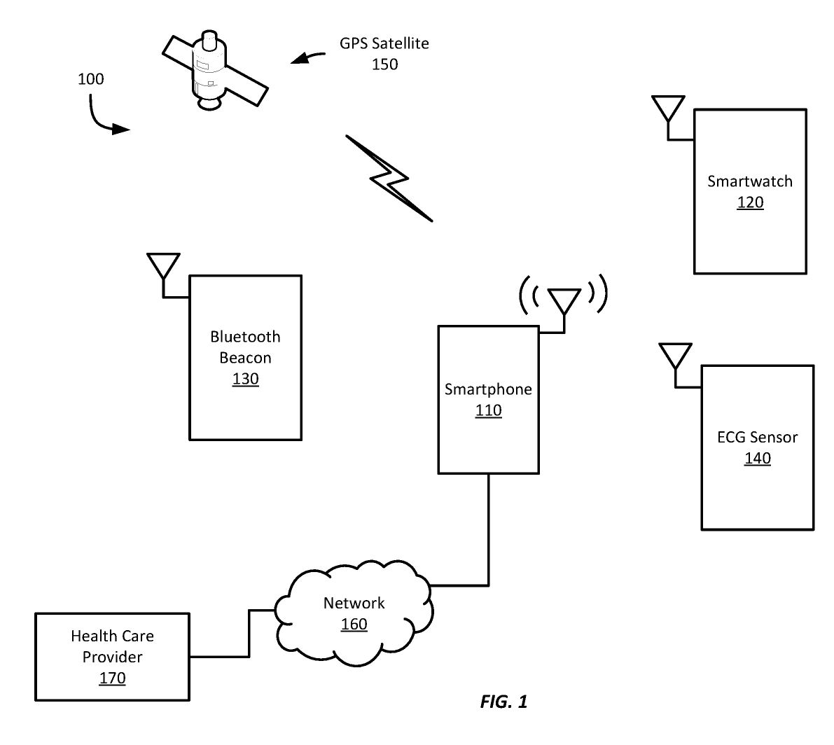

Referring now to FIG. 1, FIG. 1 shows an example system 100 for micro and macro activity detection and monitoring. The example system 100 includes a smartphone 110 that is in communication with several other wireless devices 120-140 and is receiving signals from a Global Navigation Satellite System (“GNSS”) satellite, specifically a Global Positioning System (“GPS”) satellite 150 in this example. The smartphone 110 is carried by an individual (referred to as a “patient”) and includes a software application that receives signals from various sensors or other devices, executes a ML technique to iteratively determine micro and macro activities based on the received signals, and determines the micro and macro activities that the patient is engaged in. The software application further employs one or more profiles obtained from the patient's healthcare provider, or other third party, to determine whether the patient is complying with restrictions on activity or lifestyle, performing prescribed exercise programs, etc. The software application can then provide feedback to the patient regarding whether the user is complying with the profiles or is deviating from one or more of them. In addition, the software application may, in some examples, provide notifications to one or more of the patient's health care providers based on the detected compliance or non-compliance.

In this example, the smartphone 110 receives sensor signals from the patient's smartwatch 120 and an electrocardiogram (“ECG”) sensor 140 worn by the patient. The smartphone 110 also detects a nearby BT beacon 130, which it may use to determine the patient's location with respect to known equipment within a room, such as a gym or the user's home. In addition, the smartphone 110, via the software application, is able to determine the patient's geographic position based on signals received from the GPS satellite 150.

The sensor signals received from the smartwatch 120 in this example may include pulse rate information, step count information, raw accelerometer or gyroscope information, etc. Thus, in this example, the smartphone 110 may receive raw sensor information from one or more sensors on the smartwatch, or may receive outputs of processing performed by the smartwatch 120. For example, the smartwatch 120 includes an accelerometer, which it can use to determine steps taken by the patient. It may then communicate a number of steps taken and a time period during which they were taken to the smartphone 110. In some examples, the smartwatch 120 may assign individual timestamps to each determined step and provide such information to the smartphone 110. Similarly, the smartwatch 120 may detect pulse rate using a sensor and provide time-stamped pulse rate measurements to the smartphone 110. However, in some examples, the smartwatch 120 may instead provide raw sensor information received from one or more of its sensors to the smartphone 110. In this context, “raw” sensor information refers to signals received from a sensor that are provided to the smartphone without a higher-order determination being made based on the signal and provided in lieu of the raw sensor information. It should be appreciated that “raw” sensor information, such as detected voltages, may be filtered or modified while remaining “raw” sensor information. Thus, voltages received from an accelerometer, whether filtered or not, are raw sensor information, while a step determined from the voltages is not raw sensor information.

The BT beacon and the GPS satellite may not provide direct measurements of user activity in some examples, but instead may provide context information that may be used to determine a patient's activity. For example, GPS information may be used to determine the patient's geographic position, which may then be used to determine a patient location, such as a gym, the patient's home, etc., based on the patient's geographic position. Further, a location, such as a gym, may have one or more BT beacons that can provide additional information about the patient's location. For example, a gym may position BT beacons on one or more pieces of exercise equipment, or within one or more rooms or regions of the gym. In one example, a gym may provide a BT beacon within a room having a number of metabolic conditioning equipment, such as treadmills, elliptical machines, stationary bicycles, rowing machines, etc., and provide another BT beacon within a free weight area, and a further beacon within a classroom or pool area. Thus, the smartphone 110 may be able to determine a patient's relative location within a gym to help determine an activity the patient is engaged in.

The smartphone 110 may then receive signals from the various devices 120-140 as well as the GPS satellite 150 and iteratively determine the patient's micro and macro activities, which may in turn be used to determine an activity the patient is engaged in. The information from the various devices 120-140 or the GPS satellite 150, the determined micro and macro activities, or other information may be provided to a third party, such as a health care provider 170 using network 160. The health care provider 170 may be any third party that provides health-related services to a patient, such as a doctor, nurse, therapist, personal trainer, hospital, clinic, emergency room, etc. The health care provider 170 may receive information from the smartphone 110, or it may provide information to the smartphone 110, such as one or more profiles (as will be discussed in more detail below) or one or more notifications to the patient.

It should be appreciated that the system 100 shown in FIG. 1 is only one example system 100, and other systems according to this disclosure may include a greater or smaller number of sensors or devices, or may employ different types of sensors or devices.

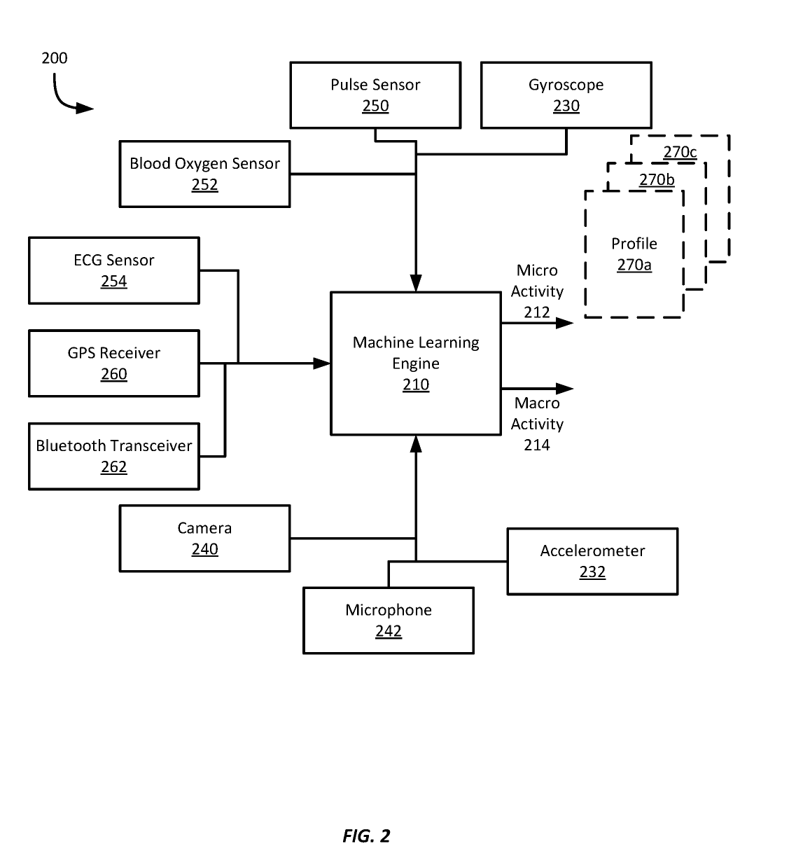

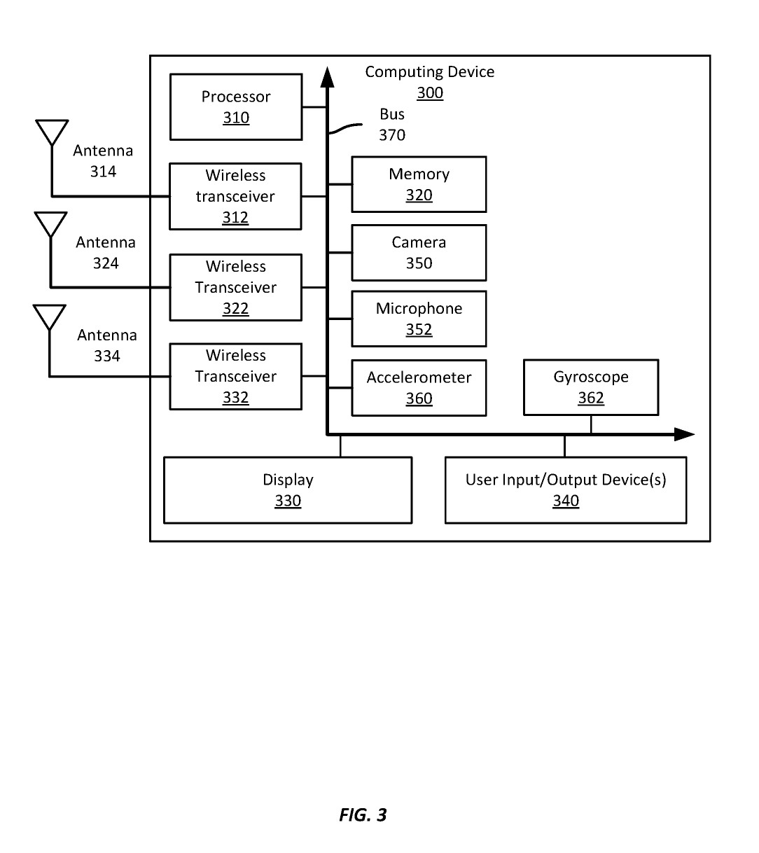

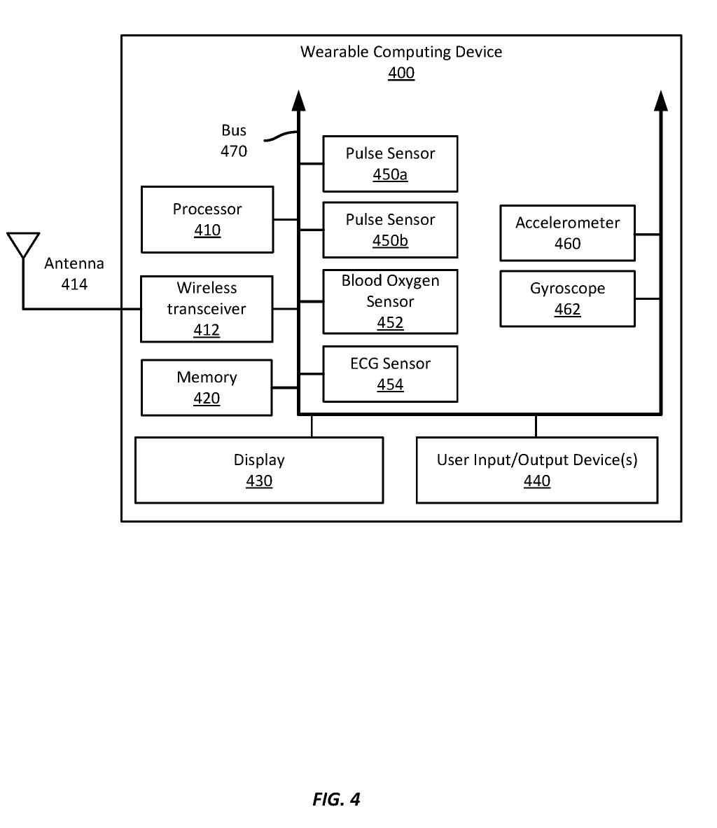

Referring now to FIG. 2, FIG. 2 shows an example system 200 for micro and macro activity detection and monitoring. The system 200 in this example, includes multiple sensors that provide sensor information to a trained ML engine 210. The trained ML engine 210 generates micro and macro activity outputs 212, 214. The system 200 may then compare the determined micro and macro activities against one or more profiles 270a-c to determine whether the patient is complying with, for example, a physical therapy program, exceeding certain thresholds, such as heartrate, intensity durations, etc. The system 200 can then output notifications to the patient indicating compliance or non-compliance, and in some examples, may provide additional feedback information, such as remedial action to take (e.g., reduce pace, resume jogging from walking, etc.). In this example, the system 200 is embodied in one or more computing devices or wearable computing devices, which will be discussed in more detail with respect to FIGS. 3-4 below.

In this example, the system 200 includes inertial sensors, including a gyroscope 230 and an accelerometer 232; environmental sensors, including a camera 240 and a microphone 242; physiological sensors, including a pulse sensor 250, a blood oxygen sensor 252, and an ECG sensor 254; and location sensors, including a GPS receiver 260 and a BT transceiver 262. Other sensors or types of sensors may be employed according to different examples. The sensors may be incorporated into one or more computing devices or may be separate and discrete from any of a patient's computing devices. For example, the patient may wear one or more ECG sensors affixed to their skin, which may then communicate via wired or wireless communications techniques with a computing devices. Thus, the system 200 show in FIG. 2 may be embodied within a single device, or may be distributed amongst multiple interconnected devices.

The various sensors shown in FIG. 2 transmit sensor information to the trained ML engine 210, though one or more of the signals may be filter or otherwise processed prior to being provided to the trained ML engine 210. For example, the pulse sensor 240 may detect a patient's pulse using any suitable pulse sensor, such as using infrared light emitters and detectors. The pulse sensor 250 may then provide to the trained ML engine 210 any related information according to different examples, such as pulse rate, individual pulses (with or without time stamps), pulse characteristics, pulse wave velocity, etc. Alternatively, the pulse sensor may provide raw output from one or more of the detectors, which may then be processed by the trained ML engine 210 or another technique to obtain the relevant pulse information. Similarly, other sensors may provide raw, filtered, or processed sensor information to the trained ML engine 210.

The ML engine 210 in this example has been trained using labelled sensor data sets obtained from activities performed by the user. For example, after the user obtains a software application embodying a ML engine and installs it on a wearable computing device, such as a smartwatch, the user may perform a training sequence for various activities to provide ground truth data for the ML engine 210. For example, the smartwatch may prompt the user to jog at low intensity for 5 minutes, then prompt the user to jog at medium intensity for 5 minutes, then prompt the user to jog at high intensity for 60 seconds. The software application may then obtain sensor information from any available sensor and train the ML technique according to the sensor information for the specified activity. These training sequences then may be repeated multiple times to acquire larger data sets. Further, in some examples, the trained ML engine 210 may continue to refine itself during use by using live data and seeking confirmation from the user regarding the type of activity being performed. Thus, over time, the trained ML engine 210 may become increasingly personalized for the user and provide increasingly-accurate micro and macro activity determination.

The trained ML engine 210 receives sensor information from one or more of the sensors and applies the sensor information to a trained ML technique associated with the patient to generate a micro activity 212 and a macro activity 214. Any suitable ML technique may be employed according to different examples. In some examples, the ML technique may assign different priorities or classifications to different types of sensor information. In one example, the trained ML technique may employ primary and secondary designations for sensor information. For example, the trained ML technique may assign inertial sensor information, e.g., from an accelerometer or gyroscopic, a high priority or weight, or mark the inertial sensor information as “primary” sensor information. Such high priority information may more substantially weight the analysis performed by the trained ML technique than lower priority information. In some examples, different weights or priorities may be assigned to different types of sensor information according to different types of possible micro or macro activities.

For example, the trained ML technique may have been trained to detect when the patient is walking on a treadmill that has a nearby BT beacon by using inertial sensor data, the BT beacon connection, and pulse information as “primary” data, while information from other sensors may be identified as “secondary” data. In one example, the trained ML technique may determine a candidate micro or macro activity only using the primary data while subsequently determining whether the secondary data corroborates or refutes the candidate micro or macro activity. For example, if the primary data includes step counts provided by an accelerometer, a connection with a known BT beacon, and a heart rate of 125 beats per minute (“bpm”), the trained ML technique may output a candidate micro activity of “walking on a treadmill.” However, if secondary information, such as GPS geographic information indicates that the user is outside in a park, the candidate micro activity may be refuted or be weighted with a reduced probability score.

In the example system 200 shown in FIG. 2, the trained ML engine 210 outputs a pair of values at a pre-configured periodic rate, e.g., once per second. The pair of values includes a determined micro activity, such as “walking,” and a determined macro activity, such as “low intensity.” The outputs from the trained ML engine 210 can then be accumulated as pairs of values over a period of time, each referred to as a “sample.” The system 200 may then aggregate the determined micro and macro activities and compare them against a profile 270a-c provided by one or more health care providers, or other third parties.

The profiles 270a-c in this example include information about recommended physical activity, such as physical therapy or exercise routines; activity thresholds, including maximum or minimum times for performing one or more activities; exertion thresholds, such as maximum heart rate, maximum sustained heartrate, maximum exertion level; or any combination of these or other information. Micro and macro activities determined by the system 200 based on the trained ML technique may then be compared against the information stored in one or more profiles to document or ensure compliance with a profile or to help ensure the patient does not put themselves at risk of overexertion.

For example, a profile may include an entry to jog at low intensity for ten minutes and the same profile, or another, may include a threshold indicating that the patient should not sustain a pulse rate exceeding 125 bpm for more than five minutes and should not exceed a pulse rate of 140 bpm at any time. Thus, as the patient begins jogging at a low intensity, the time spent jogging may be accumulated with respect to the prescribed ten minutes, while the patient's pulse rate is tracked against the two pulse rate thresholds, the limit on sustained pulse rate and the limit on instantaneous pulse rate. When the ten minutes has elapsed, the system 200 may mark the jogging exercise as complete; however, during the jogging, if the patient exceeds 125 bpm for more than 5 minutes or exceeds 140 bpm, the system 200 may output a notification to alert the patient to the exertion threshold.

Further, in some examples, the system 200 may determine when a patient is approaching an exertion threshold and output a notification. For example, if the patient has sustained a pulse rate of 130 bpm for four minutes and thirty seconds, the system 200 may output a notification indicating that the patient is nearing the five minute threshold. Similarly, if the patient's heart rate reaches 135 bpm, the system 200 may output a notification warning the patient that their pulse rate is nearing the 140 bpm threshold. Such advanced warnings may be established at a predetermined percentage of the exertion threshold. For example, an advanced warning may be output when the patient's pulse rate achieves 97% of the maximum instantaneous pulse rate. In some examples, the advanced warnings may be established at predetermined times, such as 10 seconds before reaching a maximum sustained pulse rate threshold. Still other types of relative or absolute advanced warning thresholds may be employed according to different examples.

Referring now to FIG. 3, FIG. 3 shows an example computing device 300 for micro and macro activity detection and monitoring. The example computing device 300 includes a processor 310, memory 320, a display 330, user input and output devices 340, a microphone 352, a camera 350, an accelerometer 360, and a gyroscope in communication with each other via bus 370. In addition, the computing device 300 includes three wireless transceivers 312, 322, 332 and associated antennas 314, 324, 334. The processor 310 is configured to execute processor-executable program code stored in the memory 320 to execute one or more methods for micro and macro activity detection and monitoring according to this disclosure.

In this example, the computing device 300 is a smartphone. However, the computing device may be any computing device configured to receive sensor signals and execute a trained ML technique. Example computing devices according to this disclosure may be laptop computers, desktop computers, tablets, phablets, satellite phones, cellular phones, dedicated video conferencing equipment, IOT hubs, virtual assistant device (such as Alexa®, Home®, etc.), wearable devices (such as smart watches, earbuds, headphones, Google Glass®, etc.), etc.

In this example, the smartphone 300 is equipped with a wireless transceiver 312, 322, 332 and corresponding antennas 314,324, 334 configured to wirelessly communicate using any suitable wireless technology with any device, system or network that is capable of transmitting and receiving RF signals according to any of the IEEE 16.11 standards, or any of the IEEE 802.11 standards, the BT standard, code division multiple access (CDMA), frequency division multiple access (FDMA), time division multiple access (TDMA), Global System for Mobile communications (GSM), GSM/General Packet Radio Service (GPRS), Enhanced Data GSM Environment (EDGE), Terrestrial Trunked Radio (TETRA), Wideband-CDMA (W-CDMA), Evolution Data Optimized (EV-DO), 1xEV-DO, EV-DO Rev A, EV-DO Rev B, High Speed Packet Access (HSPA), High Speed Downlink Packet Access (HSDPA), High Speed Uplink Packet Access (HSUPA), Evolved High Speed Packet Access (HSPA+), Long Term Evolution (LTE), AMPS, or other known signals that are used to communicate within a wireless, cellular or internet of things (IOT) network, such as a system utilizing 3G, 4G or 5G, or further implementations thereof, technology. In addition, one or more transceivers 312, 322, 332 may be configured as, or replaced by, a GNSS receiver, such as a GPS receiver.

The processor 310 in this example is configured to execute processor-executable program code stored in the memory 320, which may include processor-executable program code to execute a machine learning technique embodied in a machine learning engine, such as described above with respect to FIG. 2.

Referring now to FIG. 4, FIG. 4 shows an example wearable computing device 400 for micro and macro activity detection and monitoring. In this example, the wearable computing device 400 is a smartwatch; however as discussed above, the computing device 300 of FIG. 3 may be in communication with other devices, which may include any of a wide variety of electronic devices, including smartwatches, wristbands, headphones, one or more ear buds, patches worn on the skin, etc. In addition, the computing device 400 includes a wireless transceiver 412 and associated antenna 414 that may be used to communicate with the example computing device shown in FIG. 3 using any suitable wireless communications technology as discussed above.

In this example, the computing device 400 includes multiple sensors 450a-b, 452, 454, 460, 462 via bus 470 that may be used to sense one or more movements or physiological parameters of a patient. In this example, the accelerometer 460 and gyroscope 462 may be used to sense user movements, such as steps, arm swings, hand or joint orientation changes, etc. The pulse sensors 450a-b may be employed to sense heart rate, pulse wave velocity, etc. The blood oxygen sensor 452 may provide signals indicative of blood oxygen saturation, while the ECG sensor 454 may provide information indicative of heart activity.

The processor 410 may receive raw sensor information from one or more sensors and provide the raw sensor information to the computing device 300 of FIG. 3. However, in some examples, the processor 410 may perform its own processing, such as to determine steps, heart rate, pulse wave velocity etc., which may then be provided to the other computing device 300.

While the example wearable computing device 400 shown in FIG. 4 includes certain types and numbers of sensors, any suitable sensor or number of sensors may be incorporated into a wearable computing device according to this disclosure.

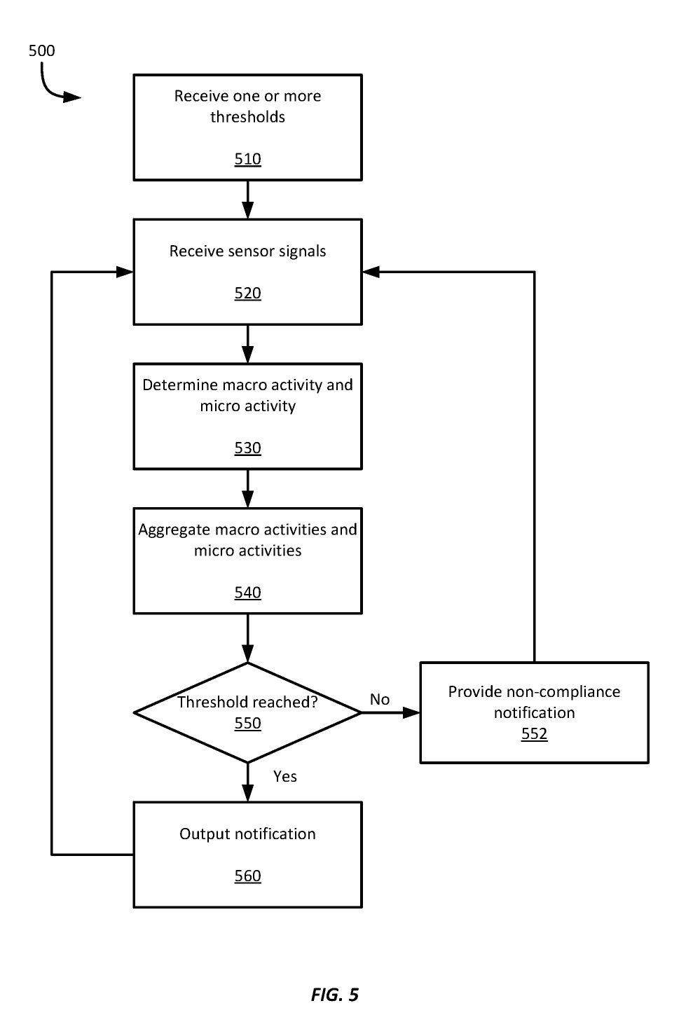

Referring now to FIG. 5, FIG. 5 shows an example method 500 for micro and macro activity detection and monitoring. The example method 500 shown in FIG. 5 will be discussed with respect to the system 200 shown in FIG. 2 and computing devices shown in FIGS. 3-4. In this example, a patient is wearing a smartwatch 400 and is carrying a smartphone 300; however, any suitable devices or systems according to this disclosure may be employed according to different examples.

At block 510, the patient's smartphone 300 receives one or more profiles from one (or more) of the patient's health care providers. In this example, the term health care provider broadly relates to anyone that provides health, wellness, or related services. A health care provider can include a doctor, nurse, physician's assistant, physical therapist, personal trainer, insurer, etc. A profile can include any suitable parameters, thresholds, or goals to be monitored for the patient. For example, a profile can include physiological thresholds, such as exercise regimens, exercise limits, exertion limits, maximum or minimum heart rates, maximum or minimum blood oxygen concentrations, etc. Physiological thresholds may also be associated with a time period, which may include a duration (e.g., 5 seconds, 10 minutes, etc.) or a single reading or measurement (e.g., 145 bpm). Further, physiological thresholds may be associated with additional information, such as warnings or notifications. In one example, a heart rate profile may indicate a maximum heart rate of 145 bpm, designate the threshold relates to a “single measurement,” that the threshold is a “do not exceed” threshold, and that a notification is to be provided to both the patient and to the health care provider that provided the profile, e.g., the patient's cardiologist. Such a profile, or profile entry, may be recorded as follows:

```
<profile>
    <sensor>pulse</sensor>
    <maximum>145</maximum>
    <duration>0</duration>
    <type>DNE</type>
    <alert>
        <target>localhost</target>
        <message>”Maximum heart rate exceeded!!
    Reduce activity level!!</message>
    </alert>
    <alert>
        <target>roberts@cardiologists.com</target>
    </alert>
</profile>
```

In this example, the threshold is established as a pulse threshold with a maximum value of 145 bpm at any time (a duration of zero in this example, refers to an instantaneous or single reading or measurement). The threshold is a Do Not Exceed (“DNE”) threshold and alerts are output to the patient's device and to the email address “roberts@cardiologists.com.” Other example thresholds may be provided according to different examples. It should be noted that many possible thresholds may be established, and many may be highly technical or otherwise indecipherable to the patient, such as thresholds related to ECG readings, blood oxygen levels, pulse wave velocities, etc. However, by configuring a profile for a patient, a health care provider may be able to remotely monitor the patient and, if needed, inform the patient of a detected condition and, in some examples, include specific messages, instructions, or warnings for the patient without requiring the patient understand the specific parameters being monitored. Further, in some examples, one or more parameters may be monitored without providing any indication to the patient that the parameters are being monitored. Instead, one or more notifications regarding such private parameters may only be provided to a health care provider.

In some examples a profile may establish an exercise or similar goal, such as prescribed by a physical therapist or personal trainer. For example, a physical therapist may provide a profile that indicates that the patient should take a minimum of 2,500 steps per day, but a maximum of 4,000 steps per day, as she recovers from a knee surgery. Further, the profile may cover a period of several weeks, during which the minimum and maximum thresholds may change. Or in some examples, the physical therapist may vary the goals daily, such as to provide recovery days or higher intensity days. One example profile is as follows:

```
<profile>
    <sensor>step</sensor>
    <minimum>2500</minimum>
    <maximum>4000</maximum>
    <duration>86400</duration>
    <type>Count</type>
    <alert>
        <type>Warning</type>
        <count>3600</count>
        <target>localhost</target>
        <message>”Approaching maximum number of
    steps!</message>
    </alert>
    <alert>
        <type>Alert</type>
        <count>4000</count>
        <target>localhost</target>
        <message>”<Maximum number of steps
    reached!</message>
        <target>j.smith@physicaltherapy.com</target>
    </alert>
</profile>
```

In this example, the thresholds established by the profile are minimum and maximum thresholds. In addition, a warning threshold of 3600 steps (90% of the maximum) is established. When the maximum threshold is reached, the smartphone 300 notifies the patient and the patient's physical therapist. In other examples, other types of profiles with different thresholds, sensor types, notifications, etc. may be established. Further, multiple profiles may be employed simultaneously, or one or more profiles may be selectively activated or deactivated.

In this example, the patient's smartphone 300 receives the profile(s), however, in some examples, the patient's smartwatch 400, may receive one or more profiles. Or some profiles may be received by the smartphone 300 and others may be received by the smartwatch 400. In some examples, the smartphone 300 may receive a profile from a health care provider and may then provide a copy of the profile to the smartwatch 400.

After one or more profiles is received, the patient's smartwatch 400 or smartphone 300 begins receiving sensor signals. In this example, blocks 520 to 560 are performed iteratively over a period of time to monitor the patient's activity and to monitor progress or conditions associated with the received profile(s).

At block 520, the patient's smartphone 300 or smartwatch 400 receives sensor signals from one or more of its sensors 450a-b, 452, 456, 460, 462. The sensor signals include information obtained by the respective sensor 450a-b, 452, 456, 460, 462 and may include raw, filtered, or processed sensor information as discussed above. In this example, the two pulse sensors 450a-b provide sensor signals that indicate a pulse. The sensor signals are transmitted every time a pulse is detected. Thus, the smartwatch's processor 410 is able to determine an approximate time at which each pulse occurs as well as the relative times each of the pulse sensors 450a-b detects a pulse. Each received pulse is timestamped and used to determine the patient's then-current pulse rate. In addition, the time differences between pulse signals received from the two pulse sensors can be used to compute a pulse wave velocity. The pulse information can then be stored by the processor 410 in the smartwatch's memory 420 or transmitted to the smartphone 300. In addition, the smartwatch 400 receives sensor signals from other sensors 452, 452, 460, 462 and obtains sensor information based on the sensor signals, such as blood oxygen concentration, ECG readings, steps taken, etc.

In some examples, the smartwatch 400 or the smartphone 300 may receive sensor signals from position sensors, such as GPS, WiFi, BT, etc. Such sensor signals may provide location information, such as geographic locations or a location within a particular building or business. For example, the patient's smartphone 300 may receive GPS signals that indicate a latitude and a longitude associated with the patient's gym. The smartphone 300 may also receive a WiFi signal from a WiFi access point (“AP”) associated with the patient's gym. In some examples, the smartphone 300 (or the smartwatch 400) may identify or connect with a BT beacon located within a building or business, and determine the patient's relative location within the building or business. Such relative location information may indicate proximity to exercise equipment, workout space, a pool, a basketball court, a tennis court, etc. Thus, sensor signals from location sensors may be obtained and employed according to different examples.

In some examples, the smartwatch 400 or smartphone 300 may receive sensor signals from environmental sensors, such as cameras 350, microphones 352, etc. Sensor signals from such sensors may be employed to determine activities performed by the patient or determine a location or context of the patient. For example, a microphone may record audio signals, which may be processed by the smartphone 300 or smartwatch 400 to detect audio features associated with relative locations, such as a pool, a treadmill, etc., or with activities, such as walking or running, etc. In some examples, the environmental sensors may record spoken words or commands from the patient. Image sensors, such as cameras, may provide images or video, which may be employed to recognize objects within the images or video, such as treadmills, weights, etc. Such environmental information may be used to further determine activities performed by the user, as will be discussed below.

Further, the environmental information may provide contextual information about the patient's then-current environmental context. Contextual information may include a location or an environment that the patient is in, such as at home, at the gym, at the office or job site, at a restaurant, outdoors, in a vehicle, etc. Such contextual information may be determined based on received sensor information, such as from a GNSS receiver, a BT beacon signal, one or more cameras, etc. The contextual information may then be provided to the trained ML engine as input information in addition to sensor information from one or more sensors.

At block 530, the smartphone 300 determines, using a trained ML technique for the individual, such as trained ML technique 210, a macro activity 214 and a micro activity 212 performed by the patient and accumulates the macro activity 214 and the micro activity 212. In this example, the trained ML technique 210 has been trained based on sensor data obtained by monitoring the patient. The training sensor data was provided to the ML technique 210 along with relevant labels to indicate the respective correct output. Thus, when the method 500 is performed, the trained ML technique 210 receives new sensor information received from the smartwatch's sensors 450a-b, 452, 454, 460-462, the smartphone's sensors 350, 352, 360, 362, position sensors, such as GPS, BT, WiFi, etc. or any combination of any of these. The sensor information may be grouped into sets of information that is periodically provided to the trained ML technique 210, or sensor information may be provided as it is received. The trained ML technique 210 may then determine a micro activity 212 and a macro activity 214 based on the received sensor information.

Different examples according to this disclosure may employ different types of micro and macro activities. Micro and macro activities for a particular patient may vary, or may be selected, based on a patient's particular traits, physical condition, chronic health conditions, etc. In this example, however, the trained ML technique can select among the following micro activities: walking, walking slowly, walking fast, walking on stairs, running, swimming, aerobic activity, typing, eating, sitting, standing, lying down, sleeping, lifting weights, etc.

In this example, the trained ML technique 210 determines a micro activity 212 and a macro activity 214 based on the received sensor signals from one or more of the sensors. As discussed above, blocks 520 to 560 may be performed iteratively over a period of time. Thus, in this example, the trained ML technique 210 determines a new micro activity and a new macro activity every second based on received sensor signals. However, in some examples the trained ML technique 210 may determine new micro and macro activities at a different rate. Suitable rates may vary according to different examples, or may be specified within one or more of the received profiles. In some examples, the determination rate may be synchronous, e.g., every second, or asynchronous, e.g., whenever new sensor signals are received.

In this example, the trained ML technique 210 may determine a micro activity, such as walking, walking quickly, walking slowly, climbing or descending stairs, jogging, running, swimming, typing, eating, sitting, standing, lying down, sleeping, etc. The types of micro activities, and which sensor inputs cause the ML technique 210 to determine a particular micro activity, will depend upon the configuration of the system 200 or the training of the ML technique 210. For example, an example trained ML technique 200 may have a set of micro activities that includes walking, but does not have different gradations of walking, such as walking quickly or walking slowly, or may not differentiate between running and jogging.

The example trained ML technique 210 can determine any of three macro activities that represent an intensity level of the determine micro activity: low intensity, moderate intensity, or high intensity. However, other examples may employ other types of macro activities or a larger number of macro activities. In one example, macro activities may include a numeric range, such as intensity level 1 through intensity level 5. In some examples, a macro activity may include additional intensities, such as stationary, resting, sleeping. For example, the trained ML technique may determine a micro activity of lying down and a macro activity of resting, if the patient is detected as being awake, or of sleeping, if the patient is detected as sleeping. Still other examples may employ additional or different sets of macro activities.

At block 540, the smartphone 300 accumulates the determined micro and macro activities. In this example, the system 200 logs each pair of determined micro and macro activities and applies a timestamp to the respective pair. For each iteration, a new micro and macro activity is determined, timestamped, and stored. Thus, over a period of time and iterations, the system 200 generates multiple determine micro activities and macro activities. For example, over a 10 second interval, the system 200 may accumulate ten pairs of timestamped micro and macro activities.

At block 550, the smartphone 300 determines whether one or more thresholds specified within a profile has been reached based on the accumulated micro and macro activities. In one example, the one or more of the individual pairs of micro and macro activities is compared against one or more thresholds within a profile or profiles to determine whether a threshold has been reached or exceeded. For example, a profile provided by a physical therapist may indicate that the patient is to walk for at least twenty-five minutes and to run for at least five minutes. The smartphone 300 may analyze the accumulated micro and macro activities to determine the amounts of time spent walking or running by the patient and compare those values against the threshold(s). If a threshold is reached, the method 500 may proceed to block 560, otherwise, the method 500 may proceed to block 552 or simply return to block 520 to obtain further sensor signals. Thus, by accumulating micro and macro activities, the smartphone 300 may determine whether the patient is complying with instructions provided by their health care provider or whether they are engaging in activities that may be dangerous or otherwise harmful to their health. Further, and as discussed above, in some examples, in addition to determining compliance with one or more profiles based on the micro and macro activities, the system 200 may also compare individual sensor signals against such thresholds to determine whether the patient is exceeding a threshold, such as pulse rate.

At block 552, the smartphone 300 may provide a non-compliance notification to the patient. For example, if a profile for the patient specifies that the patient is to perform 30 minutes of aerobic exercise per day, the smartphone 300 may monitor the patient's progress as described above, such as with respect to blocks 520-550. If the patient has not reached the exercise threshold (or goal) as determined at block 550, at block 552, the smartphone 300 may output a notification indicating that the patient has not reached the exercise threshold. For example, the smartphone 300 may output a visual notification, such as “You have only performed 10 of 30 minutes of aerobic exercise today. Please find time for additional aerobic exercise!” Such a non-compliance notification may be visual, audible, tactile, etc., or any combination thereof. For example, the smartphone 300 may output a sound, a vibration, and a visual message to notify the user of the unachieved threshold or goal.

In some examples, the smartphone 300 may not output non-compliance notifications until a predetermined time of the day. For example, if the patient has an exercise goal, it may not be desirable to provide constant or continuous notifications that the goal has not yet been met. Instead, the notification may be output at a certain time, or certain times, during the day as reminders. For example, a notification may be output shortly before lunch, e.g., at 11:30 am, in the event the user is able to take time during a lunch break to perform the required exercise. In some examples, a notification may be output after the work day, such as at 6 pm, when the user may have the opportunity to go to a gym, or to perform the exercise at home. Such notifications may be output to help remind a patient of one or more activity goals and provide sufficient opportunity to perform the prescribed activity.

In some examples, the notification may only be provided once the opportunity to perform the prescribed activity or activities has expired. For example, if profile includes a goal of a 30 minute bike ride on a particular day, if the patient fails to perform the prescribed bike ride, the smartphone 300 may output a notification the following morning indicating the missed activity goal. Thus, the smartphone 300 (or other suitable computing device) may provide to the patient feedback information regarding compliance or non-compliance with activity goals specified within one or more profiles, which may help improve the patient's rate of compliance with such goals. The method 500 may then return to block 520 to receive additional sensor signals.

At block 560, the smartphone 300 or smartwatch 400 outputs a notification regarding the threshold(s) that have been reached. Notifications may be output to the patient, to the health care provider, or to other third parties, such as family members or social media, to indicate progress with respect to one or more profiles or thresholds established by the patient's health care provider(s).

In one example, a notification may be displayed on the smartphone's 300 or smartwatch's 400 display(s) 330, 430, or a sound may be played, or a haptic effect may be output. In some examples, a notification may be sent to the health care provider associated with the threshold that was achieved. For example, if a physical therapist or personal trainer provides an exercise regimen to the patient, as the patient achieves the thresholds (or requirements) within the regimen, the smartphone 300 (or smartwatch 400) may send a notification to the physical therapist or personal trainer that indicates the threshold that was achieved. In some examples, multiple different entities may be notified, such as the patient, the health care provider, family members, friends, etc. Notifications may be provided via emails, social media posts, text or short message service (“SMS”) messages, etc.

After a notification is output at block 560, the method 500 may return to block 520 to continue obtaining sensor signals or to block 510 if a new profile is received.

It should be appreciated that the ordering of blocks shown in FIG. 5 is only one example, and that according to some examples, the blocks may be performed in different orders or simultaneously. For example, while the trained ML technique 210 is determining micro and macro activities, the system 200 may continue to receive sensor signals, or may receive a new profile, etc.

While the methods and systems herein are described in terms of software executing on various machines, the methods and systems may also be implemented as specifically-configured hardware, such as field-programmable gate array (FPGA) specifically to execute the various methods. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example computer-readable storage media, that may store instructions that, when executed by the processor, can cause the processor to perform the steps described herein as carried out, or assisted, by a processor. Examples of computer-readable media may include, but are not limited to, an electronic, optical, magnetic, or other storage device capable of providing a processor, such as the processor in a web server, with computer-readable instructions. Other examples of media comprise, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code for carrying out one or more of the methods (or parts of methods) described herein.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

What is claimed is:

1. A method comprising:
receiving a profile from a health care provider, the profile comprising a physiological threshold;
iteratively during a first time interval:
receiving first sensor signals from a first sensor, the first sensor disposed within a first device worn by an individual,
receiving second sensor signals from a second sensor, the second sensor disposed within a second device, and
determining, using a trained machine learning technique for the individual, and accumulating a macro activity and a micro activity based on the first and second sensor signals,
using the accumulated macro and micro activities, determining an aggregate macro activity and an aggregate micro activity for the first time interval; and
responsive to determining, using the aggregate macro activity and the aggregate micro activity, that the physiological threshold has been reached, outputting a notification indicating the physiological threshold.

2. The method of claim 1, further comprising transmitting the notification to the health care provider.

3. The method of claim 1, wherein the first sensor comprises (i) a pulse rate sensor, (ii) an electrocardiogram sensor, (iii) an accelerometer, (iv) a gyroscope, or (v) any combination of (i) to (iv).

4. The method of claim 1, wherein the second sensor comprises (i) a microphone, (ii) a camera, (iii) a wireless beacon, (iv) a global navigation satellite system receiver, or (v) any combination of (i) to (iv).

5. The method of claim 1, wherein the determining the macro activity and the micro activity is based on a sensed location of the individual.

6. The method of claim 5, wherein the sensed location comprises a gym, an athletic field, a track, a road, or the individual's home.

7. The method of claim 5, wherein the sensed location comprises a proximity to a piece of exercise equipment.

8. The method of claim 7, wherein the determining the aggregate macro activity and the aggregate micro activity is further based on the proximity to the piece of exercise equipment.

9. The method of claim 1, wherein the profile comprises a do-not-exceed threshold, and further comprising responsive to determining, using the aggregate macro activity and the aggregate micro activity, that the individual is within a predetermined threshold of the do-not-exceed threshold, outputting a warning notification indicating the do-not-exceed threshold.

10. The method of claim 1, wherein the first device and the second device are the same device.

11. A device comprising:
a first sensor;
a non-transitory computer-readable medium; and
a processor in communication with the first sensor and a second sensor, the processor configured to:
receive one or more profiles from a health care provider, the profile comprising a physiological threshold;
iteratively during a first time interval:
receive first sensor signals from the first sensor,
receive second sensor signals from a second sensor, and
determine, using a trained machine learning technique for an individual associated with the device, and accumulate a macro activity and a micro activity based on the first and second sensor signals,
using the accumulated macro and micro activities, determine an aggregate macro activity and an aggregate micro activity for the first time interval; and
responsive to a determination, using the aggregate macro activity and the aggregate micro activity, that the physiological threshold has been reached, output a notification indicating the physiological threshold.

12. The device of claim 11, wherein the processor is further configured to transmit the notification to the health care provider.

13. The device of claim 11, wherein the first sensor comprises (i) a pulse rate sensor, (ii) an electrocardiogram sensor, (iii) an accelerometer, (iv) a gyroscope, or (v) any combination of (i) to (iv).

14. The device of claim 11, wherein the second sensor comprises (i) a microphone, (ii) a camera, (iii) a wireless beacon, (iv) a global navigation satellite system receiver, or (v) any combination of (i) to (iv).

15. The device of claim 11, wherein the processor is further configured to determine the macro activity and the micro activity based on a sensed location of the individual.

16. The device of claim 15, wherein the sensed location comprises a gym, an athletic field, a track, a road, or the individual's home.

17. The device of claim 15, wherein the sensed location comprises a proximity to a piece of exercise equipment.

18. The device of claim 17, wherein the processor is further configured to determine the aggregate macro activity and the aggregate micro activity further based on the proximity to the piece of exercise equipment.

19. The device of claim 11, wherein the profile comprises a do-not-exceed threshold, and wherein the processor is further configured to, responsive to a determination, using the aggregate macro activity and the aggregate micro activity, that the individual is within a predetermined threshold of the do-not-exceed threshold, output a warning notification indicating the do-not-exceed threshold.

20. The device of claim 11, further comprising the second sensor.

21. A non-transitory computer-readable medium comprising processor-executable program code configured to cause a processor to:
receive one or more profiles from a health care provider, the profile comprising a physiological threshold;
iteratively during a first time interval:
receive first sensor signals from a first sensor, the first sensor disposed within a first device worn by an individual,
receive second sensor signals from a second sensor, the second sensor disposed within a second device, and
determine, using a trained machine learning technique for the individual, and accumulate a macro activity and a micro activity based on the first and second sensor signals,
using the accumulated macro and micro activities, determine an aggregate macro activity and an aggregate micro activity for the first time interval; and
responsive to a determination, using the aggregate macro activity and the aggregate micro activity, that the physiological threshold has been reached, output a notification indicating the physiological threshold.

22. The non-transitory computer-readable medium of claim 21, wherein the processor-executable program code is further configured to cause the processor to transmit the notification to the health care provider.

23. The non-transitory computer-readable medium of claim 21, wherein the first sensor comprises (i) a pulse rate sensor, (ii) an electrocardiogram sensor, (iii) an accelerometer, (iv) a gyroscope, or (v) any combination of (i) to (iv).

24. The non-transitory computer-readable medium of claim 21, wherein the second sensor comprises (i) a microphone, (ii) a camera, (iii) a wireless beacon, (iv) a global navigation satellite system receiver, or (v) any combination of (i) to (iv).

25. The non-transitory computer-readable medium of claim 21, wherein the processor-executable program code is further configured to cause the processor to determine the macro activity and the micro activity based on a sensed location of the individual.

26. The non-transitory computer-readable medium of claim 25, wherein the sensed location comprises a proximity to a piece of exercise equipment, and wherein the processor-executable program code is further configured to cause the processor to determine the aggregate macro activity and the aggregate micro activity further based on a proximity to the piece of exercise equipment.

27. The non-transitory computer-readable medium of claim 21, wherein one of the one or more profiles comprises a do-not-exceed threshold, and wherein the processor-executable program code is further configured to cause the processor to, responsive to a determination, using the aggregate macro activity and the aggregate micro activity, that the individual is within a predetermined threshold of the do-not-exceed threshold, output a warning notification indicating the do-not-exceed threshold.

28. An apparatus comprising:
means for receiving one or more profiles from a health care provider, the profile comprising a physiological threshold;
means for receiving first sensor signals from a first sensor, the first sensor disposed within a first device worn by an individual,
means for receiving second sensor signals from a second sensor, the second sensor disposed within a second device, and
means for determining, using a trained machine learning technique for the individual, and accumulating a macro activity and a micro activity based on the first and second sensor signals during a first time interval,
means for determining an aggregate macro activity and an aggregate micro activity for the first time interval using the accumulated macro and micro activities; and
means for determining, using the aggregate macro activity and the aggregate micro activity, that the physiological threshold has been reached; and
means for outputting a notification indicating the physiological threshold.

29. The apparatus of claim 28, further comprising means for transmitting the notification to the health care provider.

30. The apparatus of claim 28, wherein one of the one or more profiles comprises a do-not-exceed threshold, and further comprising:
means for determining, using the aggregate macro activity and the aggregate micro activity, that the individual is within a predetermined threshold of the do-not-exceed threshold; and
means for outputting a warning notification indicating the do-not-exceed threshold.
